(19) United States
(12) Patent Application Publication
(10) Pub. No.: US 2004/0242869 A1
(43) Pub. Date: Dec. 2, 2004

(54) Semicarbazides and their uses

(76) Inventor: David J. Carini, Wallingford, CT (US)

Correspondence Address: ROPES & GRAY LLP, ONE INTERNATIONAL PLACE, BOSTON, MA, 02110-2624, US

(21) Appl. No.: 10/482,494

(22) PCT Filed: Jul. 8, 2002
(86) PCT No.: PCT/US02/21449

Related U.S. Application Data
(60) Provisional application No. 60/303,519, filed on Jul. 6, 2001.

(51) Int. Cl. C07D 413/02; C07D 231/54; C 07D 4 3/02

(52) U.S. Cl. 544/140; 544/371; 546/199; 548/359.1

(57) ABSTRACT

The present invention relates to the synthesis of a new class of 5-substituted-3-(4-OR¹-phenyl)-2H-indeno[1,2-c]pyrazol-4-ones of formula (I):

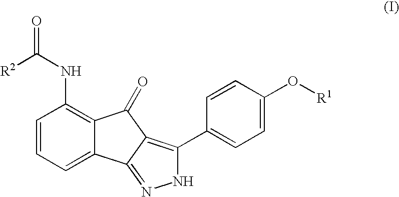

that are potent inhibitors of the class of enzymes known as cyclin dependent kinases, which relate to the catalytic subunits cdk1-7 and their regulatory subunits know as cyclins A-G.

This invention also provides a novel method of treating cancer or other proliferative diseases by administering a therapeutically effective amount of one of these compounds or a pharmaceutically acceptable salt form thereof. Alternatively, one can treat cancer or other proliferative diseases by administering a therapeutically effective combination of one of the compounds of the present invention and one or more other known anti-cancer or anti-proliferative agents.

### FIELD OF THE INVENTION

This invention relates generally to novel 5-substituted-3-(4-OR¹-phenyl)-2H-indeno[1,2-c]pyrazol-4-ones which are useful as cyclin dependent kinase (cdk) inhibitors, pharmaceutical compositions comprising the same, methods for using the same for treating proliferative diseases, and intermediates and processes for making the same.

### BACKGROUND OF THE INVENTION

One of the most important and fundamental processes in biology is the division of cells mediated by the cell cycle. This process ensures the controlled production of subsequent generations of cells with defined biological function. It is a highly regulated phenomenon and responds to a diverse set of cellular signals both within the cell and from external sources. A complex network of tumor promoting and suppressing gene products are key components of this cellular signaling process. Overexpression of the tumor promoting components or the subsequent loss of the tumor suppressing products will lead to unregulated cellular proliferation and the generation of tumors (Pardee, Science 246:603-608, 1989).

Cyclin dependent kinases (cdks) play a key role in regulating the cell cycle machinery. Cdk complexes consist of two components: ^{r}a catalytic subunit (the kinase) and a regulatory subunit (the cyclin). To date, nine kinase subunits (cdk 1-9) have been identified along with several regulatory subunits (cyclins A-H)(A. M. Senderowicz and E. A. Sausville Journal of the National Cancer Institute (2000), 92 (5), 376-387; and S. Mani; C. Wang; K. Wu; R. Francis; R. Pestell Exp. Opin. Invest. Drugs (2000) 9(8), 1849-1870). Each kinase associates with a specific regulatory partner and together make up the active catalytic moiety. Each transition of the cell cycle is regulated by a particular cdk complex: G1/S by cdk2/cyclin E, cdk4/cyclin D1 and cdk6/cyclinD2; S/G2 by cdk2/cyclin A and cdk1/cyclin A; G2/M by cdk1/B. The coordinated activity of these kinases guides the individual cells through the replication process and ensures the vitality of each subsequent generation (Sherr, Cell 73:1059-1065, 1993; Draetta, Trends Biochem. Sci. 15:378-382, 1990)

An increasing body of evidence has shown a link between tumor development and cdk related malfunctions. Over expression of the cyclin regulatory proteins and subsequent kinase hyperactivity have been linked to several types of cancers (Jiang, Proc. Natl. Acad. Sci. USA 90:9026-9030, 1993; Wang, Nature 343:555-557, 1990). More recently, endogenous, highly specific protein inhibitors of cdks were found to have a major effect on cellular proliferation (Kamb et al, Science 264:436-440, 1994; Beach, Nature 336:701-704, 1993). These inhibitors include p16^{INK4} (an inhibitor of cdk4/D1), p21^{CIP1} (a general cdk inhibitor), and p27^{KIP1} (a specific cdk2/E inhibitor). A recent crystal structure of p27 bound to cdk2/A revealed how these proteins effectively inhibit the kinase activity through multiple interactions with the cdk complex (Pavletich, Nature 382:325-331, 1996). These proteins help to regulate the cell cycle through specific interactions with their corresponding cdk complexes. Cells deficient in these inhibitors are prone to unregulated growth and tumor formation.

Protein kinases, in particular, cdk, play a role in the regulation of cellular proliferation. Therefore, cdk inhibitors can be useful in the treatment of cell proliferative disorders such as cancer, familial adenomatosis polyposis, neuro-fibromatosis, psoriasis, fungal infections, endotoxic shock, trasplantaion rejection, vascular smooth cell proliferation associated with atherosclerosis, pulmonary fibrosis, arthritis glomerulonephritis and post-surgical stenosis and restenosis (U.S. Pat. No. 6,114,365). Cdks are also known to play a role in apoptosis. Therefore cdk inhibitors, could be useful in the treatment of cancer; viral infections, for example, herpevirus, poxvirus, Epstein-Barr virus, Sindbis virus and adenovirus; prevention of AIDS development in HIV-infected individuals; autoimmune diseases, for example, systemic lupus, erythematosus, autoimmune mediated glomerulonephritis, rheumatoid arthritis, psoriasis, inflammatory bowel disease, and autoimmune diabetes mellitus; neurodegenerative disorders, for example, Alzheimer's disease, AIDS-related dementia, Parkinson's disease, amyotrophic lateral sclerosis, retinitis pigmentosa, spinal muscular atrophy and cerebellar degeneration; myelodysplastic syndromes, aplastic anemia, ischemic injury associated with myocardial infarctions, stroke and reperfusion injury, arrhythmia, atherosclerosis, toxin-induced or alcohol related liver diseases, hematological diseases, for example, chronic anemia and aplastic anemia; degenerative diseases of the musculoskeletal system, for example, osteoporosis and arthritis, aspirin-sensitive rhinosinusitis, cystic fibrosis, multiple sclerosis, kidney diseases and cancer pain (U.S. Pat. No. 6,107,305).

It has also been discovered that some cyclin-dependent kinase inhibitors can be used in combination therapy with some other anticancer agents. For example, the cytotoxic activity of the cyclin-dependent kinase inhibitor, flavopiridol, has been used with other anticancer agents in cancer combination therapy. (Cancer Research, 57, 3375 (1997)).

Also, it has recently been disclosed that cdk inhibitors may be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse (U.S. Pat. No. 6,107,305).

It has recently been discovered that cdk5 is involved in the phosphorylation of tau protein, and therefore cdk inhibitors may be useful in the treatment of Alzheimer's disease (J. Biochem., 117, 741-749, 1995).

This body of evidence has led to an intense search for small molecule inhibitors of the cdk family as an approach to cancer chemotherapy.

A series of indeno[1,2-c]pyrazoles having anticancer activity are described in JP 60130521 and JP 62099361 with the following generic structure:

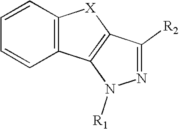

A series of indeno[1,2-c]pyrazoles having herbicidal activity are described in GB 2223946 with the following generic structure:

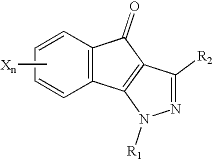

A series of 1-(6′-substituted-4′-methylquinol-2′-yl)-3-methylindeno[1,2-c]pyrazoles having CNS activity are described by Quraishi, Farmaco 44:753-8, 1989 with the following generic structure:

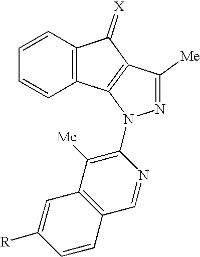

There remains a strong unmet need for new cdk inhibitors for use in treating proliferative diseases associated with cdk activity.

### SUMMARY OF THE INVENTION

The present invention describes a novel class of 5-substituted-3-(4-OR¹-phenyl)-2H-indeno[1,2-c]pyrazol-4-ones or pharmaceutically acceptable salt forms thereof that are potent inhibitors of the class of enzymes known as cyclin dependent kinases, which relate to the catalytic subunits cdk 1-9 and their regulatory subunits know as cyclins A-H.

The present invention is directed to compounds of formula (I), or pharmaceutically acceptable salts thereof, which act as cyclin dependent kinase inhibitors:

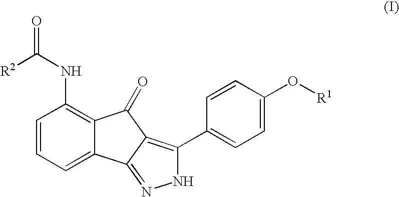

wherein:

R¹ is selected from the group consisting of —H and —C₁₋₄alkyl;

R² is selected from the group consisting of —C₁₋₄alkoxy, —NR³R⁴, and —(CH₂)NR³R⁴;

R³ is selected from the group consisting of —H and morpholino;

R⁴ is selected from the group consisting of —H and cyclohexyl

substituted with —NH₂; alternatively, R³ and R⁴ together form a 6-membered heterocycle containing 1 to 2 heteroatoms selected from nitrogen and oxygen wherein said 6-membered heterocycle is substituted with 1 R⁵; and

R⁵ is selected from the group consisting of —H, —NH₂, —CH₂NH₂, and —CH₂CH₂NH₂.

The present invention is also directed to a novel method of treating proliferative diseases associated with CDK activity by administering a therapeutically effective amount of one of the compounds of the present invention or a pharmaceutically acceptable salt form thereof to a patient in need of such therapy.

The present invention also relates to a novel method of treating cancer associated with CDK activity by administering a therapeutically effective amount of one of the compounds of the invention or a pharmaceutically acceptable salt form thereof.

A novel method of treating a proliferative disease, which comprises administering a therapeutically effective combination of one of the compounds of the present invention in combination with one or more other known anti-cancer treatments such as radiation therapy, chemotoxic or chemostatic agents is also dislosed.

### DETAILED DESCRIPTION OF THE INVENTION

Compounds of the present invention have formula (I), or pharmaceutically acceptable salts thereof, which act as cyclin dependent kinase inhibitors:

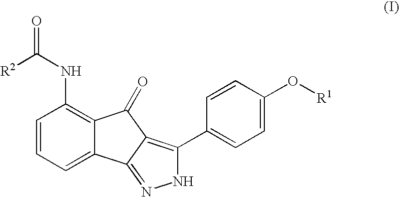

wherein

R¹ is selected from the group consisting of —H and —C₁₋₄alkyl;

R² is selected from the group consisting of —C₁₋₄alkoxy, —NR³R⁴, and —(CH₂)NR³R⁴;

R³ is selected from the group consisting of —H and morpholino;

R⁴ is selected from the group consisting of —H and cyclohexyl

substituted with —NH₂; alternatively, R³ and R⁴ together form a 6-membered heterocycle containing 1 to 2 heteroatoms selected from nitrogen and oxygen wherein said 6-membered heterocycle is substituted with 1 R⁵; and

R⁵ is selected from the group consisting of —H, —NH₂, —CH₂NH₂, and —CH₂CH₂NH₂.

As used above, and throughout the description of the invention, the following terms, unless otherwise indicated, shall be understood to have the following meaning. The term “compounds of the invention”, and equivalent expressions, are meant to embrace compounds of formula (I), and includes prodrugs, pharmaceutically acceptable salts, and solvates, e.g. hydrates. Similarly, reference to intermediates, whether or not they themselves are claimed, is meant to embrace their salts, and solvates, where the context so permits.

The term “derivative” means a chemically modified compound wherein the modification is considered routine by the ordinary skilled chemist, such as an ester or an amide of an acid, protecting groups, such as a benzyl group for an alcohol or thiol, and tert-butoxycarbonyl group for an amine.

The term “analogue” means a compound which comprises a chemically modified form of a specific compound or class thereof, and which maintains the pharmaceutical and/or pharmacological activities characteristic of said compound or class.

The term “solvate” means a physical association of a compound of this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, and the like.

The term “effective amount” means an amount of a compound/composition according to the present invention effective in producing the desired therapeutic effect. The term “patient” includes both human and other mammals. The term “pharmaceutical composition” means a composition comprising a compound of formula (I) in combination with at least one additional pharmaceutical adjuvant, excipient, vehicle and/or carrier component pharmaceutically acceptable, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Any ingredient listed in Remington's Pharmaceutical Sciences, 18^{th} ed., Mack Publishing Company, may be used.

The term “alkyl” is intended to include both branched and straight-chain saturated aliphatic hydrocarbon groups having the specified number of carbon atoms. Examples of alkyl include, but are not limited to, methyl, ethyl, n-propyl, i-propyl, n-butyl, s-butyl, and t-butyl.

The term “alkoxy” is intended to represent an alkyl group with the indicated number of carbon atoms attached to an oxygen atom. Examples of alkoxy include, but are not limited to, methoxy, ethoxy, n-propoxy, i-propoxy, n-butoxy, s-butoxy, and t-butoxy.

As used herein, the term “heterocycle” or “heterocyclic system” means a cyclic compound which consists of carbon atoms and from 1 to 2 heteroatoms independently selected from the group consisting of nitrogen and oxygen atoms. The nitrogen atom may optionally be oxidized. The heterocyclic ring may be attached to its pendant group at any heteroatom or carbon atom which results in a stable structure. The heterocyclic rings described herein may be substituted on carbon or on a nitrogen atom if the resulting compound is stable. If specifically noted, a nitrogen in the heterocycle may optionally be quaternized.

Examples of heterocycles include, but are not limited to piperidinyl, morpholinyl, or piperazinyl groups.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p. 1445, the disclosure of which is hereby incorporated by reference.

The compounds of the present invention are useful in the form of the free base or acid or in the form of a pharmaceutically acceptable salt thereof. All forms are within the scope of the invention.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable risk/benefit ratio.

The term “pharmaceutically acceptable prodrugs” as used herein means those prodrugs of the compounds useful according to the present invention which are, within the scope of sound medical judgment, suitable for use in contact with the tissues of humans and lower animals without undue toxicity, irritation, allergic response, and the like, commensurate with a reasonable risk/benefit ratio, and effective for their intended use, as well as zwitterionic forms, where possible, of the compounds of the invention.

The term “prodrugs”, as the term is used herein, are intended to include any covalently bonded carriers which release an active parent drug of the present invention in vivo when such prodrug is administered to a mammalian subject. Since prodrugs are known to enhance numerous desirable qualities of pharmaceuticals (i.e., solubility, bioavailability, manufacturing, etc.) the compounds of the present invention may be delivered in prodrug form. Thus, the skilled artisan will appreciate that the present mention encompasses prodrugs of the presently claimed compounds, methods of delivering the same, and compositions containing the same. Prodrugs of the present invention are prepared by modifying functional groups present in the compound in such a way that the modifications are cleaved, either in routine manipulation or in vivo, to the parent compound. The transformation in vivo may be, for example, as the result of some metabolic process, such as chemical or enzymatic hydrolysis of a carboxylic, phosphoric or sulphate ester, or reduction or oxidation of a susceptible functionality. Prodrugs include compounds of the present invention wherein a hydroxy, amino, or sulfhydryl group is bonded to any group that, when the prodrug of the present invention is administered to a mammalian subject, it cleaves to form a free hydroxyl, free amino, or free sulfydryl group, respectively. Functional groups which may be rapidly transformed, by metabolic cleavage, in vivo form a class of groups reactive with the carboxyl group of the compounds of this invention. They include, but are not limited to such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds useful according to this invention are cleaved in vivo, the compounds bearing such groups can act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion of prodrugs is provided in the following: Design of Prodrugs, H. Bundgaard, ed., Elsevier, 1985; Methods in Enzymology, K. Widder et al, Ed., Academic Press, 42, p.309-396, 1985; A Textbook of Drug Design and Development, Krogsgaard-Larsen and H. Bundgaard, ed., Chapter 5; “Design and Applications of Prodrugs” p.113-191, 1991; Advanced Drug Delivery Reviews, H. Bundgard, 8, p.1-38, 1992; Journal of Pharmaceutical Sciences, 77, p. 285, 1988; Chem. Pharm. Bull., N. Nakeya et al, 32, p. 692, 1984; Pro-drugs as Novel Delivery Systems, T. Higuchi and V. Stella, Vol. 14 of the A.C.S. Symposium Series, and Bioreversible Carriers in Drug Design, Edward B. Roche, ed., American Pharmaceutical Association and Pergamon Press, 1987, each of which is herein incorporated by reference in their entirety as though set forth in full.

The term “treating” refers to: (i) preventing a disease, disorder or condition from occurring in an animal which may be predisposed to the disease, disorder and/or condition but has not yet been diagnosed as having it; (ii) inhibiting the disease, disorder or condition, i.e., arresting its development; and (iii) relieving the disease, disorder or condition, i.e., causing regression of the disease, disorder and/or condition.

### Preparation of Compounds of the Invention

It will be apparent to those skilled in the art that certain compounds of formula (I) can exhibit isomerism, for example geometrical isomerism, e.g., E or Z isomerism, and optical isomerism, e.g., R or S configurations. Geometrical isomers include the cis and trans forms of compounds of the invention having alkenyl moieties. It is well known in the art how to prepare optically active forms, such as by resolution of racemic forms or by synthesis from optically active starting materials. All chiral, diastereomeric, racemic forms and all geometric isomeric forms of a structure are intended, unless the specific stereochemistry or isomer form is specifically indicated.

Such isomers can be separated from their mixtures, by the application or adaptation of known methods, for example chromatographic techniques and recrystallization techniques, or they are separately prepared from the appropriate isomers of their intermediates, for example by the application or adaptation of methods described herein.

Where the compound of the present invention is substituted with a basic moiety, acid addition salts are formed and are simply a more convenient form for use; and in practice, use of the salt form inherently amounts to use of the free base form. The acids which can be used to prepare the acid addition salts include preferably those which produce, when combined with the free base, pharmaceutically acceptable salts, that is, salts whose anions are non-toxic to the patient in pharmaceutical doses of the salts, so that the beneficial inhibitory effects on CDK inherent in the free base are not vitiated by side effects ascribable to the anions. Although pharmaceutically acceptable salts of said basic compounds are preferred, all acid addition salts are useful as sources of the free base form even if the particular salt, per se, is desired only as an intermediate product as, for example, when the salt is formed only for purposes of purification, and identification, or when it is used as an intermediate in preparing a pharmaceutically acceptable salt by ion exchange procedures.

According to a further feature of the invention, acid addition salts of the compounds of this invention are prepared by reaction of the free base with the appropriate acid, by the application or adaptation of known methods. For example, the acid addition salts of the compounds of this invention are prepared either by dissolving the free base in aqueous or aqueous-alcohol solution or other suitable solvents containing the appropriate acid and isolating the salt by evaporating the solution, or by reacting the free base and acid in an organic solvent, in which case the salt separates directly or can be obtained by concentration of the solution.

The acid addition salts of the compounds of this invention can be regenerated from the salts by the application or adaptation of known methods. For example, parent compounds of the invention can be regenerated from their acid addition salts by treatment with an alkali, e.g. aqueous sodium bicarbonate solution or aqueous ammonia solution.

Where the compound of the invention is substituted with an acidic moiety, base addition salts may be formed and can be simply a more convenient form for use; and in practice, use of the salt form can inherently amounts to use of the free acid form. The bases which can be used to prepare the base addition salts include those which produce, when combined with the free acid, pharmaceutically acceptable salts, that is, salts whose cations are non-toxic to the animal organism in pharmaceutical doses of the salts, so that the beneficial inhibitory effects on CDK inherent in the free acid are not vitiated by side effects ascribable to the cations. Pharmaceutically acceptable salts, including for example alkali and alkaline earth metal salts, within the scope of the invention are those derived from the following bases: sodium hydride, sodium hydroxide, potassium hydroxide, calcium hydroxide, aluminum hydroxide, lithium hydroxide, magnesium hydroxide, zinc hydroxide, ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, and the like.

Metal salts of compounds of the present invention may be obtained by contacting a hydride, hydroxide, carbonate or similar reactive compound of the chosen metal in an aqueous or organic solvent with the free acid form of the compound. The aqueous solvent employed may be water or it may be a mixture of water with an organic solvent, preferably an alcohol such as methanol or ethanol, a ketone such as acetone, an aliphatic ether such as tetrahydrofuran, or an ester such as ethyl acetate. Such reactions are normally conducted at ambient temperature but they may, if desired, be conducted with heating.

Amine salts of compounds of the present invention may be obtained by contacting an amine in an aqueous or organic solvent with the free acid form of the compound. Suitable aqueous solvents include water and mixtures of water with alcohols such as methanol or ethanol, ethers such as tetrahydrofuran, nitrites such as acetonitrile, or ketones such as acetone. Amino acid salts may be similarly prepared.

The base addition salts of the compounds of this invention can be regenerated from the salts by the application or adaptation of known methods. For example, parent compounds of the invention can be regenerated from their base addition salts by treatment with an acid, e.g. hydrochloric acid.

Pharmaceutically acceptable salts also include quaternary lower alkyl ammonium salts. The quaternary salts are prepared by the exhaustive alkylation of basic nitrogen atoms in compounds, including nonaromatic and aromatic basic nitrogen atoms, according to the invention, i.e., alkylating the non-bonded pair of electrons of the nitrogen moieties with an alkylating agent such as methylhalide, particularly methyl iodide, or dimethyl sulfate. Quaternarization results in the nitrogen moiety becoming positively charged and having a negative counter ion associated therewith.

As will be self-evident to those skilled in the art, some of the compounds of this invention do not form stable salts. However, acid addition salts are more likely to be formed by compounds of this invention having a nitrogen-containing heteroaryl group and/or wherein the compounds contain an amino group as a substituent. Preferable acid addition salts of the compounds of the invention are those wherein there is not an acid labile group.

As well as being useful in themselves as active compounds, salts of compounds of the invention are useful for the purposes of purification of the compounds, for example by exploitation of the solubility differences between the salts and the parent compounds, side products and/or starting materials, by techniques well known to those skilled in the art.

Compounds according to the invention, for example, starting materials, intermediates or products, are prepared as described herein or by the application or adaptation of known methods, by which is meant methods used heretofore or described in the literature, for example those described by R. C. Larock in Comprehensive Organic Transformations, VCH publishers, 1989.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Green and P. G. M. Wuts in “Protective Groups in Organic Chemistry” John Wiley and Sons, 1991; J. F. W. McOmie in “Protective Groups in Organic Chemistry” Plenum Press, 1973.

The compounds useful according to the invention optionally are supplied as salts. Those salts which are pharmaceutically acceptable are of particular interest since they are useful in administering the foregoing compounds for medical purposes. Salts which are not pharmaceutically acceptable are useful in manufacturing processes, for isolation and purification purposes, and in some instances, for use in separating stereoisomeric forms of the compounds of this invention. The latter is particularly true of amine salts prepared from optically active amines. Where the compound useful according to the invention contains a carboxy group, or a sufficiently acidic bioisostere, base addition salts may be formed and are simply a more convenient form for use; and in practice, use of the salt form inherently amounts to use of the free acid form.

Also, where the compound useful according to the invention contains a basic group, or a sufficiently basic bioisostere, acid addition salts may be formed and are simply a more convenient form for use; and in practice, use of the salt form inherently amounts to use of the free base form.

The foregoing compounds useful according to the invention may also be combined with another therapeutic compound to form pharmaceutical compositions (with or without diluent or carrier) which, when administered, provide simultaneous administration of two or more active ingredients resulting in the combination therapy of the invention.

While it is possible for compounds useful according to the invention to be administered alone it is preferably to present them as pharmaceutical compositions. The pharmaceutical compositions, both for veterinary and for human use, useful according to the present invention comprise at lease one compound of the invention, as above defined, together with one or more acceptable carriers therefor and optionally other therapeutic ingredients. The skilled artisan will appreciate the abundance of publications setting forth the state of the art for pharmaceutical administration.

Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monosterate and gelatin. Examples of suitable carriers, diluents, solvents or vehicles include water, ethanol, polyols, suitable mixtures thereof, vegetable oils (such as olive oil) and injectable organic esters such as ethyl oleate. Examples of excipients include lactose, milk sugar, sodium citrate, calcium carbonate, dicalcium phosphate phosphate. Examples of disintegrating agents include starch, alginic acids and certain complex silicates. Examples of lubricants include magnesium stearate, sodium lauryl sulphate, talc, as well as high molecular weight polyethylene glycols.

In certain preferred embodiments, active ingredients necessary in combination therapy may be combined in a single pharmaceutical composition for simultaneous administration.

The choice of vehicle and the content of active substance in the vehicle are generally determined in accordance with the solubility and chemical properties of the active compound, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulphate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used.

The oily phase of the emulsions of this invention may be constituted from known ingredients in a known manner. While the oily phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the emulsifying wax, and the way together with the oil and fat make up the emulsifying ointment base which forms the oily dispersed phase of a cream formulation. Emulgents and emulsion stabilizers suitable for use in the formulation of the present invention include Tween® 60, Span® 80, cetostearyl alcohol, benzyl alcohol, myristyl alcohol, glyceryl mono-stearate and sodium lauryl sulfate.

If desired, the aqueous phase of the cream base may include, for example, a least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogues.

The choice of suitable oils or fats for the formulation is based on achieving the desired cosmetic properties. Thus the cream should preferably be a non-greasy, non-staining and washable product with suitable consistency to avoid leakage from tubes or other containers. Straight or branched chain, mono- or dibasic alkyl esters such as di-isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used. Solid compositions may also be employed as fillers in soft and hard-filled gelatin capsules using such excipients as lactose or milk sugar as well as high molecular weight polyethylene glycols, and the like.

The pharmaceutical compositions can be administered in a suitable formulation to humans and animals by topical or systemic administration, including oral, inhalational, rectal, nasal, buccal, sublingual, vaginal, parenteral (including subcutaneous, intramuscular, intravenous, intradermal, intrathecal and epidural), intracisternal and intraperitoneal. It will be appreciated that the preferred route may vary with for example the condition of the recipient.

The formulations can be prepared in unit dosage form by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

A tablet may be made by compression or moulding, optionally with one or more accessory ingredients. Compressed tables may be prepared by compressing in a suitable machine the active ingredient in a free-flowing form such as a powder or granules, optionally mixed with a binder, lubricant, inert diluent, preservative, surface active or dispersing agent. Moulded tablets may be made by moulding in a suitable machine a mixture of the powdered compounds moistened with an inert liquid diluent. The tablets may optionally be coated or scored and may be formulated so as to provide slow or controlled release of the active ingredient therein.

Solid compositions for rectal administration include suppositories formulated in accordance with known methods and containing at least one compound of the invention.

If desired, and for more effective distribution, the compounds can be microencapsulated in, or attached to, a slow release or targeted delivery systems such as a biocompatible, biodegradable polymer matrices (e.g. poly(d,l-lactide co-glycolide)), liposomes, and microspheres and subcutaneously or intramuscularly injected by a technique called subcutaneous or intramuscular depot to provide continuous slow release of the compound(s) for a period of 2 weeks or longer. The compounds may be sterilized, for example, by filtration through a bacteria retaining filter, or by incorporating sterilizing agents in the form of sterile solid compositions which can be dissolved in sterile water, or some other sterile injectable medium immediately before use.

Actual dosage levels of active ingredient in the compositions of the invention may be varied so as to obtain an amount of active ingredient that is effective to obtain a desired therapeutic response for a particular composition and method of administration. The selected dosage level therefore depends upon the desired therapeutic effect, on the route of administration, on the desired duration of treatment and other factors.

Total daily dose of the compounds useful according to this invention administered to a host in single or divided doses may be in amounts, for example, of from about 0.0001 to about 100 mg/kg body weight daily and preferably 0.01 to 10 mg/kg/day. Dosage unit compositions may contain such amounts of such submultiples thereof as may be used to make up the daily dose. It will be understood, however, that the specific dose level for any particular patient will depend upon a variety of factors including the patient's body weight, general health, sex, diet, time and route of administration, rates of absorption and excretion, combination with other drugs and the severity of the particular disease being treated.

The amount of each component administered is determined by the attending clinicians taking into consideration the etiology and severity of the disease, the patient's condition and age, the potency of each component and other factors.

The formulations may be presented in unit-dose or multi-dose containers, for example sealed ampoules and vials with elastomeric stoppers, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Administration of a compound of the present invention in combination with additional therapeutic agents, may afford an efficacy advantage over the compounds and agents alone, and may do so while permitting the use of lower doses of each. A lower dosage minimizes the potential of side effects, thereby providing an increased margin of safety. The combination of a compound of the present invention with such additional therapeutic agents is preferably a synergistic combination. Synergy, as described for example by Chou and Talalay, Adv. Enzyme Regul. 22:27-55 (1984), occurs when the therapeutic effect of the compound and agent when administered in combination is greater than the additive effect of either the compound or agent when administered alone. In general, a synergistic effect is most clearly demonstrated at levels that are (therapeutically) sub-optimal for either the compound of the present invention or a known anti-proliferative agent alone, but which are highly efficacious in combination. Synergy can be in terms of improved inhibitory response without substantial increases in toxicity over individual treatments alone, or some other beneficial effect of the combination compared with the individual components.

Procedures for evaluating the biological activity of compounds or compositions according to the invention are carried out as described herein or by the application or adaptation of known procedures, by which is meant procedures used heretofore or as described in the literature. The compounds of the present invention, their methods or preparation and their biological activity will appear more clearly from the examination of the following examples which are presented as an illustration only and are not to be considered as limiting the invention in its scope. The following examples are but preferred methods of synthesizing the compounds of the invention, which may be prepared according to any method known to the organic chemist of ordinary skill. Other features of the invention will become apparent during the following descriptions of exemplary embodiments which are given for illustration of the invention and are not intended to be limiting thereof. Each of the cited references are hereby incorporated herein by reference in their entirity as though set forth in full.

### EXAMPLES

The following abbreviations are used throughout the following Examples: “°C” for degrees Celsius, “CIMS” for chemical ionization mass spectroscopy, “eq” for equivalent or equivalents, “g” for gram or grams, “h” for hour or hours, “mg” for milligram or milligrams, “mL” for milliliter or milliliters, “mmol” for millimolar, “M” for molar, “min” for minute or minutes, “p-TsOH” for para-toluenesulphonic acid, “DMF” for dimethylformamide, and “TFA” for trifluoroacetic acid.

### Example 1

### Preparation of Intermediate 1

The preparation of intermediate 1, (N-[2-(4-Methoxy-benzoyl)-1,3-dioxo-indan-4-yl]-acetamide) is described in Nugiel, D. A.; Etzkorn, A. M.; Vidwans, A.; Benfield, P. A.; Boisclair, M.; Burton, C. R.; Cox, S.; Czerniak, P. M.; Doleniak, D.; Seitz, S. P. J. Med. Chem. 2001, 44, 1334-1336 which is herein incorporated by reference in it's entirety as though set forth in full.

### Example 2

### Preparation of Intermediate 2

Synthesis of 4-Amino-2-(4-methoxy-benzoyl)-indan-1,3-dione: The compound prepared in example 1 (2.0 g, 5.93 mmol) is dissolved in 20% HCl in methanol (50 mL). This solution is stirred at reflux for a period of 3 h. It is then allowed to cool to room temperature and stirred overnight. The product is filtered off, washed with ethanol (20 mL) and air dried to give the product as a yellow solid (1.5 g, 85.7%). mp 268-269° C.; ¹H NMR (DMSOd₆) δ 8.17 (d, J=8.8 Hz, 2H), 7.49 (t, 1H), 7.12 (d, J=8.7 Hz, 2H), 6.98 (m, 2H), 3.88 (s, 1H).

### Example 3

### Preparation of Intermediate 3

Synthesis of [2-(4-Methoxybenzoyl)-1,3-dioxo-indan-4-yl]-carbamic acid phenyl ester: The product prepared in Example 2 (1.5 g, 5.08 mmol) is dissolved in acetone (40 mL) and treated with sodium carbonate (1.26 g, 15.24 mmol) and phenyl chloroformate (1.19 g, 7.62 mmol). The suspension is stirred at 50° C. for 3 h. The reaction mixture is diluted with water (120 mL), and extracted with ethyl acetate (2×100 mL). The organic layer is separated, washed with brine (50 mL), dried (Na₂SO₄) and the solvent removed at reduced pressure to give a gummy orange residue. Cold ethyl ether (100 mL) is added to this residue to give a precipitate. The precipitate is collected and washed with ethyl ether (2×10 mL) to give desired product as a yellow solid (1.65 g. 78%). mp 256-258° C.; ¹HNMR (DMSOd₆) δ 10.83 (s, 1H), 8.08 (d, J=8.0 Hz, 1H), 7.57 (d, J=2.9 Hz, 2H), 7.54 (m, 3H), 7.28 (m, 3H), 7.09 (t, 1H), 6.89 (d, J=10.8 Hz, 2H), 3.81 (s, 3H)

### Example 4

### Preparation of 1-[3-(4-methoxy-phenyl)-4-oxo-2,4-dihydro-indeno[1,2-c]pyrazol-5-yl]-3-morpholin-4-yl-urea

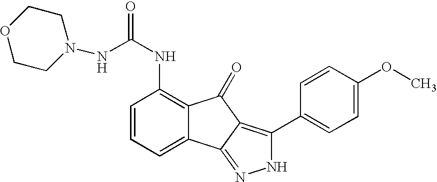

The product prepared in Example 3 (0.03 g, 0.072 mmol) in anhydrous DMSO (2 mL) is treated with 4-aminomorpholine (0.0084 g, 0.082 mmol) and 4-dimethylaminopyridine (0.005 g, 0.04 mmol) and heated to 80° C. for 3 h. The solvent is removed under reduced pressure and the residue triturated with ethanol to give a dark solid. The solid is collected and washed with ethanol (5 mL) to give a tricarbonyl urea (0.03 g, 100%). The tricarbonyl urea intermediate (0.03 g, 0.078 mmol) is treated with hydrazine hydrate (0.1 mL, 3.21 mmol) and p-toluenesulfonic acid monohydrate (0.01 g, 0.05 mmol) in refluxing ethanol (4 mL) for a period of 3 h. The reaction mixture is cooled to room temperature, the solid collected, washed with cold ethanol (2×2 mL), and air dried to give the product as a yellowish solid (0.012 g, 41.3%). mp 290-291° C.; ¹H NMR (DMSO-d₆) δ 8.27 (d, J=6.8 Hz, 2H), 8.16 (d, J=8.8 Hz, 2H), 7.42 (m, 1H), 7.12 (m, 3H), 3.81 (s, 3H), 2.90 (s, 4H), 2.70 (s, 4H), HRMS calcd. for C₂₂H₂₂N₅O₄ (M+H⁺) 420.1672; found 420.1688;

### Example 5

### Preparation of [3-(4-methoxy-phenyl)-4-oxo-2,4-dihydro-indeno[1,2-c]pyrazol-5-yl]-urea

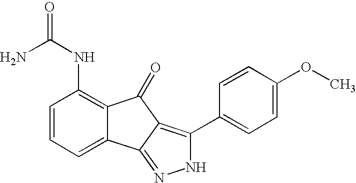

The product prepared in Example 3 (0.03 g, 0.072 mmol) in anhydrous DMSO (2 mL) is treated with excess ammonium hydroxide solution and 4-dimethylaminopyridine (0.005 g, 0.04 mmol) and is heated to 80° C. for 3 h. The solvent is removed under reduced pressure and the residue triturated with ethanol to give a dark solid. The solid is collected and washed with ethanol (5 mL) to give urea (0.03 g, 100%). The tricarbonyl urea intermediate (0.03 g, 0.078 mmol) is treated with hydrazine hydrate (0.1 mL, 3.21 mmol) and p-toluenesulfonic acid monohydrate (0.01 g, 0.05 mmol) in refluxing ethanol (4 mL) for a period of 3 h. The reaction mixture is cooled to room temperature, the solid collected, washed with cold ethanol (2×2 mL), and air dried to give the product as a yellowish solid (0.018 g, 62.4%). mp 267-269° C.; ¹H NMR (DMSO-d₆) δ 9.35 (s, 1H), 8.22 (m, 3H), 7.38 (m, 1H), 7.10 (d, J=8.8 Hz, 2H), 7.02 (d, J=0.7 Hz, 1H), 3.81 (s, 3H); HRMS calcd. for C₁₈H₁₅N₄O₃ (M+H⁺) 335.1144; found 335.1162;

### Example 6

### Preparation of 1-(2-amino-cyclohexyl)-3-[3-(4-methoxy-phenyl)-4-oxo-2,4-dihydro-indeno[1,2-c]pyrazol-5-yl]-urea

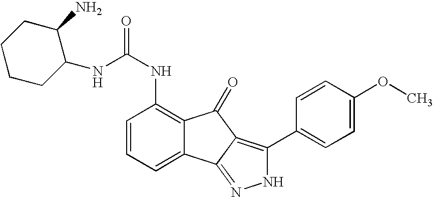

The product prepared in Example 3 (0.03 g, 0.072 mmol) in anhydrous DMSO (2 mL) is treated with 1,2-diaminocyclohexane (0.01 g, 0.082 mmol) and 4-dimethylaminopyridine (0.005 g, 0.04 mmol) and heated to 80° C. for 3 h. The solvent is removed under reduced pressure and the residue triturated with ethanol to give a dark solid. The solid is collected and washed with ethanol (5 mL) to give a tricarbonyl urea (0.03 g, 100%). The tricarbonyl urea intermediate (0.03 g, 0.078 mmol) is treated with hydrazine hydrate (0.1 mL, 3.21 mmol) and p-toluenesulfonic acid monohydrate (0.01 g, 0.05 mmol) in refluxing ethanol (4 mL) for a period of 3 h. The reaction mixture is cooled to room temperature, the solid collected, washed with cold ethanol (2×2 mL), and air dried to give the product as a yellowish solid (0.01 g, 30.6%). ¹HNMR (DMSO-d₆) δ 9.56 (s, 1H), 8.27 (d, 1H), 8.19 (d, 2H), 7.41 (t, 1H), 7.10 (m, 3H), 4.10 (s, 1H), 3.81 (s, 3H), 3.23 (s, 1H), 1.63 (m, 5H), 1.40 (m, 3H).

### Example 7

### Preparation of 5-Amino-3-(4-methoxyphenyl)-2-phenyl-2H-indeno-[1,2-c]pyrazol-4-one:

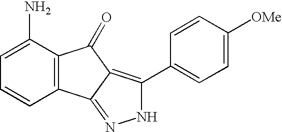

A suspension of N-[3-(4-Methoxy-phenyl)-4-oxo-2,4-dihydro-indeno[1,2-c]pyrazol-5-yl]-acetamide (as produced according to Nugiel, D. A.; Etzkorn, A. M.; Vidwans, A.; Benfield, P. A.; Boisclair, M.; Burton, C. R.; Cox, S.; Czerniak, P. M.; Doleniak, D.; Seitz, S. P. J. Med. Chem. 2001, 44, 1334-1336) (1.0 g, 3.0 mmol) in MeOH (10 mL) was treated with concentrated HCl (1 mL) and heated to reflux. After stirring the mixture for 2 h the reaction was cooled and the product was collected by filtration and obtained as a greenish solid (0.7 g, 81%). mp 273° C.; NMR (DMSO-d) δ 13.6 (bs, 1H), 8.3 (d, J=8.4 Hz, 1H), 8.1 (d, J=8.8 Hz, 2H), 7.5 (t, J=7.7 Hz 1H), 7.2 (d, J=7.0 Hz, 1H), 7.1 (d, J=8.8 Hz, 2H), 3.8 (s, 3H); HRMS m/e calc'd for C₁₇H₁₄N₃O₂ (M+H): 292.1086, found: 292.1080.

### Example 8

### Preparation of 2-Chloro-N-[3-(4-methoxyphenyl)-4-oxo-2,4-dihydro-indeno[1,2-c]pyrazol-5-yl]-acetamide:

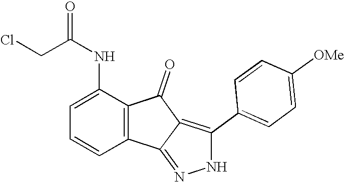

A suspension of the product prepared in Example 7 (0.2 g, 0.7 mmol) in dioxane (10 mL) was treated with aqueous' saturated NaHCO₃ (3 mL) and chloroacetyl chloride (3 mL, 0.21 mmol). The reaction was heated to 50° C. and stirred for 2 h. The reaction is then cooled, poured into water (20 mL), extracted with EtOAc (100 mL), the organic layer separated, dried (MgSO₄) and the solvent removed at reduced pressure. The residue is recrystallized from EtOH to give the product as a yellow solid (0.09 g, 35%). mp>300° C.; NMR (DMSO-d₆) δ 13.6 (bs, 1H), 11.3 (s, 1H), 8.3 (d, J=8.4 Hz, 1H), 8.1 (d, J=8.8 Hz, 2H), 7.5 (t, J=7.7 Hz 1H), 7.2 (d, J=7.0 Hz, 1H), 7.1 (d, J=8.8 Hz, 2H), 4.5 (s, 2H), 3.8 (s, 3H); HRMS m/e calc'd for C₁₉H₁₅N₃O₃Cl (M+H): 368.0802, found: 368.0818.

### Example 9

### Preparation of 2-(4-aminomethyl-piperidin-1-yl)-N-[3-(4-methoxy-phenyl)-4-oxo-2,4-dihydro-indeno[1,2-c]pyrazol-5-yl]-acetamide

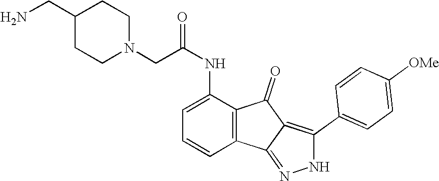

A suspension of product prepared according to Example 8 (0.015 g, 0.04 mmol) in EtOH (1 mL) is treated with 4-aminomethylpiperdine (0.75 mL), placed in a sealed tube and heated to 80° C. for 3 h. The reaction is cooled and the solvent removed at reduced pressure. The residue is recrystallized from EtOH to give the product as a yellow solid (0.009 g, 62%).mp>300° C.; NMR (DMSO-d₆) δ 13.6 (bs, 1H), 11.3 (s, 1H), 8.35 (d, J=8.4 Hz, 1H), 8.1 (d, J=8.8 Hz, 2H), 7.5 (t, J=7.7 Hz 1H), 7.2 (d, J=7.0 Hz, 1H), 7.1 (d, J=8.8 Hz, 2H), 3.8 (s, 3H), 3.2 (bs, 2H), 2.9(bs, 2H), 2.5 (d, J=8.0 Hz, 2H), 2.2 (t, J=8.0 Hz, 2H), 1.6 (m, 5H); HRMS m/e calc'd for C₂₅H₂₈N₅O₃ (M+H): 446.2192, found: 446.2169; Anal. (C₂₅H₂₇N₅O₃) C, H, N.

### Example 10

### Preparation of 2-(4-Methoxybenzoyl)-3-methoxycarbonylamino-indan-1,3-dione:

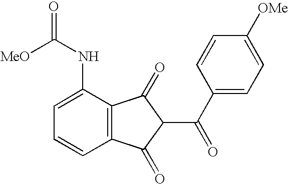

A solution of 3-methoxycarbonylamino-phthalic acid dimethyl ester (1 g, 4.8 mmol) and 4-methoxyacetophenone (0.72 g, 4.8 mmol) in dry DMF (3 mL) was heated to 90° C. Sodium hydride (0.21 g, 60% suspension in oil, 5.2 mmol) is added in one portion and the exothermic reaction turns deep red. After 20 min, the reaction is cooled to room temperature, diluted with water (25 mL) extracted with EtOAc (10 mL) and the aqueous phase separated. The aqueous phase is acidified to pH 2 with 2N HCl and the crude product collected. Recrystallization with ethanol gives the desired product as a yellow solid (0.4 g, 30%). ESIMS 352 (M−H, 100%).

### Example 11

### Preparation of 3-(4-Methoxyphenyl)-5-methoxycarbonylamino-2H-indeno-[1,2-c]pyrazol-4-one:

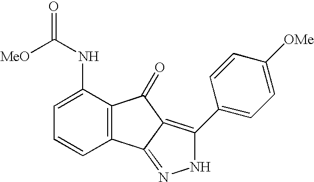

A solution of 2-(4-methoxybenzoyl)-3-methoxycarbonylamino-indan-1,3-dione (0.2 g, 0.6 mmol) in EtOH (5 mL) is treated with hydrazine hydrate (0.1 mL, 1.8 mmol) and p-TsOH (3 mg). The reaction is heated to reflux and stirred for 2 h. The reaction is cooled to room temperature and the product crystallized from the reaction mixture. The product is collected by filtration as a yellow solid (0.1 g, 50%). mp>300° C.; HRMS m/e calc'd for C₁₉H₁₆N₃O₄ (M+H): 350.1141, found: 350.1168.

### Utility

Inhibition of Kinase/Cyclin Complex Enzymatic Activity

Several of the compounds disclosed in this invention were assayed for their inhibitory activity against cdk4/D1 and cdk2/E kinase complexes. The in vitro assays employ cell lysates from insect cells expressing either of the kinases and subsequently their corresponding regulatory units. The cdk2/cyclin E is purified from insect cells expressing His-tagged cdk2 and cyclin E. The cdk/cyclin lysate is combined in a microtitre-type plate along with a kinase compatible buffer, ³²P-labeled ATP at a concentration of 50 mM, a GST-Rb fusion protein and the test compound at varying concentrations. The kinase reaction is allowed to proceeded with the radiolabled ATP, then effectively stopped by the addition of a large excess of EDTA and unlabeled ATP. The GST-Rb labeled protein is sequestered on a GSH-Sepharose bead suspension, washed, resuspended in scintillant, and the ³²p activity detected in a scintillation counter. The compound concentration which inhibits 50% of the kinase activity was calculated for each compound. A compound was considered active if its IC₅₀ was found to be less than 1 μM.

### Inhibition of HCT 116 Cancer Cell Proliferation

To test the cellular activity of several compounds disclosed in this invention, we examined the effect of these compounds on cultured HCT116 cells and determined their effect on cell-cycle progression by the calorimetric cytotoxcity test using sulforhodamine B (Skehan et al. J. Natl. Cancer Inst. 82:1107-12, 1990). Briefly, HCT116 cells are cultured in the presence of test compounds at increasing concentrations. At selected time points, groups of cells are fixed with trichloroacetic acid and stained with sulforhodamine B (SRB). Unbound dye was removed by washing and protein-bound dye was extracted for determination of optical density. A compound was considered active if its IC₅₀ was found to be less than 10 μM.

All patents, patent applications and other publications are herein incorporated by reference in their entirity as though set forth in full.

The scope of the following claims is intended to encompass all obvious changes in the details, materials and synthesis that will occur to one of ordinary skill in the art.

What is claimed is:

1. A compound of formula (I): 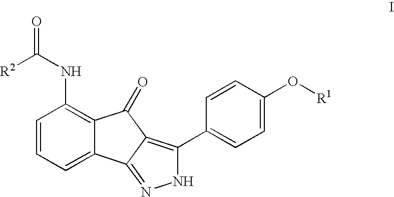
stereoisomers thereof, N-oxides thereof, pharmaceutically acceptable salts thereof, or prodrugs thereof, wherein:
R¹ is selected from the group consisting of —H and —C_{1-C}₄ alkyl;
R² is selected from the group consisting of —C₁-C₄ alkoxy, —NR³R⁴, and —(CH₂)NR³R⁴;
R³ is selected from the group consisting of —H and morpholino;
R⁴ is selected from the group consisting of —H and cyclohexyl
substituted with —NH₂; alternatively, R³ and R⁴ together form a 6-membered heterocycle containing 1 to 2 heteroatoms selected from nitrogen and oxygen wherein said 6-membered heterocycle is optionally substituted with 1 R⁵; and
R⁵ is selected from the group consisting of —H, —NH₂, —CH₂NH₂, and —CH₂CH₂NH₂.

2. A compound of claim 1, wherein:
R¹ is selected from the group consisting of H, methyl, ethyl, and propyl;
R² is selected from the group consisting of C₁-C₄ alkoxy, —NR³R⁴, or —(CH₂)NR³R⁴;
R³ is selected from the group consisting of H and morpholino;
R⁴ is selected from the group consisting of H or cyclohexyl substituted with —NH₂; alternatively, R³ and R⁴ together form a 6-membered heterocycle containing 1 to 2 heteroatoms selected from nitrogen and oxygen wherein said 6-membered heterocycle is substituted with 1 R⁵; and
R⁵ is selected from the group consisting of —CH₂NH₂ and —CH₂CH₂NH₂.

3. A compound of claim 1, wherein:
R¹ is selected from the group consisting of methyl and ethyl;
R² selected from the group consisting of —OCH₃, —OCH₂CH₃, —NR³R⁴, or —(CH₂)NR³R⁴;
R³ is selected from the group consisting of H or morpholino;
R⁴ is selected from the group consisting of H or cyclohexyl substituted with —NH₂; and alternatively, R³ and R⁴ together form a piperidinyl substituted by 1 R⁵; and,
R⁵ is —CH₂NH₂.

4. A compound of claim 1, stereoisomers thereof, N-oxides thereof, pharmaceutically acceptable salts thereof, and prodrugs thereof, selected from:
a) 1-[3-(4-methoxy-phenyl)-4-oxo-2,4-dihydro-indeno[1,2-c]pyrazol-5-yl]-3-morpholin-4-yl-urea;
b) [3-(4-methoxy-phenyl)-4-oxo-2,4-dihydro-indeno[1,2-c]pyrazol-5-yl]-urea;
c) 1-(2-amino-cyclohexyl)-3-[3-(4-methoxy-phenyl)-4-oxo-2,4-dihydro-indeno[1,2-c]pyrazol-5-yl]-urea; and
d) 2-(4-aminomethyl-piperidin-1-yl)-N-[3-(4-methoxy-phenyl)-4-oxo-2,4-dihydro-indeno[1,2-c]pyrazol-5-yl]-acetamide.

5. A compound of claim 1 consisting of 1-[3-(4-methoxy-phenyl)-4-oxo-2,4-dihydro-indeno[1,2-c]pyrazol-5-yl]-3-morpholin-4-yl-urea, stereoisomers thereof, N-oxides thereof, pharmaceutically acceptable salts thereof, or prodrugs thereof.

6. A compound of claim 1 consisting of [3-(4-methoxy-phenyl)-4-oxo-2,4-dihydro-indeno[1,2-c]pyrazol-5-yl]-urea, stereoisomers thereof, N-oxides thereof, pharmaceutically acceptable salts thereof, or prodrugs thereof.

7. A compound of claim 1 consisting of 1-(2-amino-cyclohexyl)-3-[3-(4-methoxy-phenyl)-4-oxo-2,4-dihydro-indeno[1,2-c]pyrazol-5-yl]-urea, stereoisomers thereof, N-oxides thereof, pharmaceutically acceptable salts thereof, or prodrugs thereof.

8. A compound of claim 1 consisting of 2-(4-aminomethyl-piperidin-1-yl)-N-[3-(4-methoxy-phenyl)-4-oxo-2,4-dihydro-indeno[1,2-c]pyrazol-5-yl]-acetamide, stereoisomers thereof, N-oxides thereof, pharmaceutically acceptable salts thereof, or prodrugs thereof.

9. A compound of claim 1 consisting [3-(4-methoxy-phenyl)-4-oxo-2,4-dihydro-indeno[1,2-c]pyrazol-5-yl]-carbamic acid methyl ester, stereoisomers thereof, N-oxides thereof, pharmaceutically acceptable salts thereof, or prodrugs thereof.

10. A pharmaceutical composition, comprising a pharmaceutically acceptable carrier together with a compound according to claim 1 or a pharmaceutically acceptable salt or prodrug form thereof.

11. A pharmaceutical composition comprising a compound of Formula I according to claim 1 and a pharmaceutically acceptable excipient.

12. A method of inhibiting cdk activity in a patient in need of such treatment comprising the steps of administering to said patient a theraputically effective amount of a compound according to claim 1.
